(19) United States
(12) Patent Application Publication
(10) Pub. No.: US 2017/0295821 A1
(43) Pub. Date: Oct. 19, 2017

(54) ISOLATION OF SOLUBLE PROTEINS FROM AGGREGATED CASEIN-CONTAINING MIXTURES

(71) Applicant: Upfront Chromatography A/S, Copenhagen Ø (DK)

(72) Inventors: Allan Otto Fog Lihme, Farum (DK); Marie Bendix Hansen, Frederiksberg (DK)

(73) Assignee: Upfront Chromatography A/S, Copenhagen Ø (DK)

(21) Appl. No.: 15/517,009

(22) PCT Filed: Oct. 6, 2015
(86) PCT No.: PCT/DK2015/000039

§ 371 Date: Apr. 5, 2017

(30) Foreign Application Priority Data
Oct. 6, 2014 (DK) PA 2014 00568
Nov. 21, 2014 (DK) PA 2014 00679

(51) Int. Cl. A23J 1/20; B01D 15/38; A23C 9/146; A23C 19/02; C07K 14/47; C07K 1/16

(52) U.S. Cl. CPC A23J 1/20; C07K 14/47; C07K 1/165; A23C 9/1465; A23C 19/02; B01D 15/3847

(57) ABSTRACT

The present invention relates to a method for separating at least one soluble protein fraction from an aggregated casein-containing material, the method comprises the steps of: (i) providing the aggregated casein-containing material; (ii) Contacting the aggregated casein-containing material with a chromatographic support allowing one or more soluble protein(s) present in the aggregated casein-containing material to be retained by the chromatographic support; (iii) Obtaining a permeate fraction from the chromatographic support comprising aggregated casein; (iv) Optionally washing the chromatographic support; (v) Subjecting the chromatographic support to at least one elution buffer obtaining at least one soluble protein fraction from the chromatographic support; and wherein the chromatographic support comprises one or more mixed-mode ligands capable of binding the soluble proteins from the aggregated casein-containing material.

### TECHNICAL FIELD OF THE INVENTION

The present invention relates to a method for separating soluble proteins directly from aggregated casein-containing materials, such as milk. In particular, the present invention relates to the separation of one or more soluble protein(s) by subjecting an aggregated casein-containing material to a chromatographic support capable of binding specifically to the soluble protein(s).

### BACKGROUND OF THE INVENTION

Milk is a very complex material and industrial processes use milk to produce casein, whey, lactose, condensed milk, powdered milk, and many other food-additives and industrial products. Milk comprises a mixture of components, such as proteins, minerals, fat, sugars, salts, and vitamins. In particular, the proteins in milk, which are mainly found as casein proteins or whey proteins, have gained increasingly attention over the years. The reason for this increased interest lies in the diversity of milk proteins and because each protein has unique attributes to nutritional, biological, functional and food ingredient applications. Furthermore, these proteins constitute, together with e.g. peptides and enzymes in milk, a major and important health and nutritional role in humans and animals.

To achieve highest possible potential of proteins and to explore or exploit the potentially functional and bioactive properties of proteins, e.g. proteins in milk, it is important to isolate native proteins by a procedure that avoids possible denaturing conditions (such as, high salt conditions, high or low pH conditions, heat or protease treatment/exposure).

The main protein component in milk is casein. Casein in milk is mainly found as micellar casein, formed by macromolecular casein aggregates. Micellar casein are porous hydrophobic structures and they have a natural tendency to aggregate and precipitate, however in milk this tendency is prevented by (a) the presence of glycomacropeptides in the casein aggregated structure and by (b) a negative charge of the micellar structure.

Casein aggregation and precipitation may be facilitated by enzymatic modification of the micellar structure, using rennet, or by acid precipitation. Enzymatic modification results in the specific hydrolyses of casein micellar whereby glycomacropeptide is lost. When micellar caseins are cleaved the micellar entities aggregates and when sufficient amounts of micellar caseins are cleaved, the aggregated casein proteins precipitate and are separated from the whey proteins as a solid fraction.

Traditionally, treatment of milk generally consist of an initial extraction of casein, such as by precipitation of aggregated micellar casein, e.g. by enzymatic modification using rennet or by acid treatment, providing a precipitate of aggregated casein, a curd, and a liquid whey protein solution.

However, this treatment is facing some disadvantages because the enzymatic modification or the acidic treatment may cause the aggregated casein and/or part of the soluble proteins to be partly degraded and the proteins may lose some of the biological activity. Furthermore, the precipitated casein may entrap some soluble proteins within the aggregate and thereby reducing the yield of soluble proteins or increase impurities in the aggregated casein precipitate.

Hence, an improved method for fractionating milk and providing soluble protein fractions which solves the above mentioned issues and which would be suitable for industrial application, would be advantageous. In particular a more efficient, specific and/or reliable method for providing a soluble protein fraction in high yields and/or purities directly from aggregated casein-containing material, such as milk, would be advantageous.

### SUMMARY OF THE INVENTION

Accordingly, it is an object of the present invention to provide an improved method for fractionating aggregated casein-containing materials into various components, such as protein fractions (various soluble protein fractions and/or insoluble protein fractions), carbohydrate fractions, mineral fractions, lipid fractions and/or water fractions. The method may be fast, cost effective and results in high quality fractions having high purity, high recovery and/or high yield.

In particular, it is an object of the present invention to provide a method for fractionating aggregated casein-containing material that solves the above mentioned problems of the prior art.

Thus, an object of the present invention relates to a method for separating at least one soluble protein fraction from an aggregated casein-containing material, the method comprises the steps of:
(i) Providing the aggregated casein-containing material;
(ii) Contacting the aggregated casein-containing material with a chromatographic support allowing one or more soluble protein(s) present in the aggregated casein-containing material to be retained by the chromatographic support;
(iii) Obtaining a permeate fraction from the chromatographic support comprising aggregated casein;
(iv) Optionally washing the chromatographic support;
(v) Subjecting the chromatographic support to at least one elution buffer obtaining at least one soluble protein fraction from the chromatographic support; and
   wherein the chromatographic support comprises one or more mixed-mode ligands capable of binding the soluble proteins from the aggregated casein-containing material.

Another aspect of the present invention relates to a soluble protein fraction obtained by a method according to the present invention.

Yet another aspect of the present invention is to provide a soluble protein fraction comprising two or more soluble proteins, wherein the two or more soluble proteins has a protein profile substantially similar to a protein profile for said soluble proteins in an aggregated casein-containing material.

In the present context, the term “protein profile” relates to the relative concentration ratio (on weight/weight basis) between two or more soluble proteins in an aggregated casein-containing material or in at least one soluble protein fraction.

Still another aspect of the present invention relates to the use of a chromatographic support comprising a porous organic polymeric base matrix having one or more mixed-mode ligands covalently attached for the separating at least one soluble protein fraction from an aggregated casein-containing material, said one or more mixed-mode ligands comprise a hydrophobic moiety and a non-aromatic nitrogen moiety.

An even further aspect of the present invention relates to the use of a soluble protein fraction according to the present invention, as an ingredient, preferably, as an ingredient in a food product, in a feed product, in a dietary product, in a pharmaceutical product, a nutraceutical product, in a therapeutic product, in a beverage product, in a skin care product; a cosmetic product; a product for nutritional immunotherapy; or a product for passive immunization.

Yet another aspect of the present invention is to provide a soluble protein fraction comprising a protein profile substantially similar to a protein profile for an aggregated casein-containing material in respect of at least immunoglobulin and alpha-lactalbumin and/or beta-lactoglobulin.

Still another aspect of the present invention relates to the use of a chromatographic support comprising one or more mixed-mode ligands for binding at least the soluble proteins immunoglobulin G or beta-casein and at least one of alpha-lactalbumin and/or beta-lactoglobulin from an aggregated casein-containing material.

An even further aspect of the present invention relates to the use of a soluble protein fraction according to the present invention as an ingredient, preferably, as an ingredient in a food product, a beverage product or a cosmetic product.

The present invention will now be described in more detail in the following.

### BRIEF DESCRIPTION OF THE FIGURES

FIG. 1 shows the efficiency of the method of the present invention to isolate a single soluble protein directly from an aggregated casein-containing material, such as skimmed milk, using mixed mode ligands. The chromatographic support may retain substantially all the soluble proteins from skimmed milk, and the individual soluble proteins may subsequently be eluted from the chromatographic support providing individual soluble protein fraction at high purity, high recovery and high yields. FIG. 1 illustrates the high purity of the beta-lactoglobulin fraction obtained by the present invention. The dashed line illustrates the beta-lactoglobulin fraction obtained directly from the chromatographic support whereas the solid line demonstrates the beta-lactoglobulin fraction obtained after an additional step of micro filtration.

The present invention will now be described in more detail in the following.

### DETAILED DESCRIPTION OF THE INVENTION

Over the years, the interest in fractionation of aggregated casein-containing materials, such as milk and providing applications for said protein fractions have enlarged. Traditionally the prior art describes fractionation of e.g. milk, starting with the extraction of casein, in particular by precipitating aggregated casein, in order to obtain a clear separation of the soluble parts from the insoluble parts. The inventors of the present invention surprisingly found a method for fractionating aggregated casein-containing materials, such as milk, on industrial scale, without the need for initial precipitation of aggregated casein, which is efficient, specific and/or reliable method for providing a soluble protein fraction in high yields and/or purities directly from said aggregated casein-containing material.

Thus, one aspect of the present invention relates to method for separating at least one soluble protein fraction from an aggregated casein-containing material, the method comprises the steps of:
(i) Providing the aggregated casein-containing material;
(ii) Contacting the aggregated casein-containing material with a chromatographic support allowing one or more soluble protein(s) present in the aggregated casein-containing material to be retained by the chromatographic support;
(iii) Obtaining a permeate fraction from the chromatographic support comprising aggregated casein;
(iv) Optionally washing the chromatographic suppo
(v) Subjecting the chromatographic support to at least one elution buffer obtaining at least one soluble protein fraction from the chromatographic support; and
   wherein the chromatographic support comprises one or more mixed-mode ligands capable of binding the soluble proteins from the aggregated casein-containing material.

A further aspect of the present invention relates to a method for separating at least one soluble protein fraction from an aggregated casein-containing material, the method comprises the steps of:
(i) providing the aggregated casein-containing material;
(ii) Contacting the aggregated casein-containing material with a chromatographic support allowing at least the soluble proteins immunoglobulin G or beta-casein and at least one of alpha-lactalbumin and/or beta-lactoglobulin present in the aggregated casein-containing material to be retained by the chromatographic support;
(iii) Obtaining a permeate fraction from the chromatographic support comprising aggregated casein;
(iv) Optionally washing the chromatographic support;
(v) Subjecting the chromatographic support to at least one elution buffer obtaining at least one soluble protein fraction from the chromatographic support; and
   wherein the chromatographic support comprises one or more mixed-mode ligands capable of binding the soluble proteins from the aggregated casein-containing material.

An even further aspect of the present invention relates to a method for separating at least one soluble protein fraction from an aggregated casein-containing material, the method comprises the steps of:
(i) Providing the aggregated casein-containing material;
(ii) Contacting the aggregated casein-containing material with a chromatographic support allowing one or more soluble protein(s) present in the aggregated casein-containing material to be retained by the chromatographic support;
(iii) Obtaining a permeate fraction from the chromatographic support comprising aggregated casein;
(iv) Optionally washing the chromatographic support;
(v) Subjecting the chromatographic support to at least one elution buffer obtaining at least one soluble protein fraction from the chromatographic support; and
   wherein the chromatographic support comprises a porous organic polymeric base matrix having one or more mixed-mode ligands covalently attached, said one or more mixed-mode ligands comprise a hydrophobic moiety and a non-aromatic nitrogen moiety.

In the present context, the term “at least one soluble protein fraction” relates to the soluble protein fraction(s) obtained by the present invention and comprising, either an individual soluble protein or in various combinations of two or more soluble proteins in the same soluble protein fraction. In the present context, the term “individual soluble protein” relates to a protein profile which has been shifted from being very much similar to the protein profile of the aggregated casein-containing material to a protein profile where at least one of the soluble proteins is favoured.

In the present context, the term “soluble protein” relates to free flowing non-aggregated constituents dissolved in the aggregated casein-containing material. Contrary to the soluble protein, the term “aggregated” relates to a structure that some very large molecules, such as casein, form when dispersed in a solvent, such as water. Such large molecules are considered to be too large to be truly soluble in water. Instead, these large molecules forms structures that allow them to remain suspended in water as if they are soluble. The dispersion of these large structures in water is known as a colloidal suspension. The structures that allow large molecules to remain colloidally suspended in water are termed micelles.

In an embodiment of the present invention, the one or more soluble protein(s) present in the aggregated casein-containing material may have a molecular size of less than 1000 kilo-Dalton (kDa), such as less than 750 kDa, such as less than 500 kDa, e.g. less than 400 kDa, such as less than 300 kDa, e.g. less than 250 kDa.

In the present context the term “separating” relates to the process of converting a mixture of substances, such as an aggregated casein-containing material, into two or more distinct fractions, at least one of the of distinct fractions is enriched in one or more substances from the mixture.

In an embodiment of the present invention, the separation relates to a process of providing at least one soluble protein fraction comprising a mixture of soluble proteins obtained from the aggregated casein-containing material. In an embodiment of the present invention the mixture of soluble proteins may be a mixture of at least 2 or more soluble proteins, such as at least 3 or more soluble proteins, e.g. at least 4 or more soluble proteins, such as at least 5 or more soluble proteins, e.g. at least 6 or more soluble proteins.

In another embodiment of the present invention, the separation relates to the provision of two or more distinct soluble protein fractions comprising an individual soluble protein, such as 3 or more distinct soluble protein fractions, e.g. 4 or more distinct soluble protein fractions, such as 5 or more distinct soluble protein fractions, e.g. 6 or more distinct soluble protein fractions.

The proteins in milk may have distinct physicochemical properties, which may influence fractionation and which may advantageously be used in order to provide a selective separation of one soluble protein fraction from another soluble protein fraction or from aggregated casein in an aggregated casein-containing material.

Such selective separation of one or more soluble protein(s) using chromatographic supports can be operated under two conditions:
a) Selective elution of one or more soluble protein(s) from the chromatographic support, or
b) Selective adsorption of one or more soluble protein(s) to the chromatographic support

In selective elution, all or a group of the soluble proteins in the aggregated casein-containing material are captured simultaneously by the chromatographic support. The chromatographic support may be rinsed from contaminants and un-captured matter. Then the soluble proteins captured are eluted one by one using specially designed elution buffers suitable for the particular proteins to be isolated. Thus, by using the selective elution technique it is possible to obtain several purified protein fractions from the same chromatographic support.

In selective adsorption, the process conditions may be optimised to capture one soluble protein over another. When the soluble protein of interest may be captured, the chromatographic support may be rinsed from contaminants followed by elution of the specific soluble protein.

In an embodiment of the present invention, the method is a selective elution process. In this way it is possible to provide two or more, such as 3 or more, e.g. 4 or more, such as 5 or more, e.g. 6 or more distinct soluble protein fractions comprising individual soluble proteins.

In present context, the term “selective adsorption” relates to a process where the chromatographic support is designed and/or where the process conditions are designed to favour binding of one component rather than another component from the aggregated casein-containing material.

In present context, the term “selective elution” relates to a process where the elution buffer is designed and/or where the process conditions are designed to favour elution of one retained soluble protein after another from the chromatographic support.

In the present context, the term “retained” relates to the act of holding or keeping the one or more soluble protein(s) in a particular place, namely in the chromatographic support. The soluble protein may be retained in the chromatographic support until the conditions are changed and the retained proteins are liberated and eluted from the chromatographic support, either simultaneously or sequentially.

In an embodiment of the present invention the method for separating the at least one soluble protein fraction from the aggregated casein-containing material may be a medium size scale production or large-scale production.

In an embodiment of the present invention, the method for separating the at least one soluble protein fraction from the aggregated casein-containing material may be a batch process or a continuous process.

### Aggregated Casein-Containing Material

According to the method described by the present invention the initial step of the method relates to providing an aggregated casein-containing material, step (i).

In an embodiment of the present invention, the aggregated casein-containing material may be obtained from any milk producing animal, and preferably animals traditionally used for large-scale milk production. Preferably, the aggregated casein-containing material is obtained from a ruminant animal, such as cattle, goats, sheep, giraffes, yaks, deer, camels, llamas or antelope.

In the present context, the term “aggregated casein-containing material” relates to a material that has not been subjected to casein precipitation by the addition of rennet, acid, heat or any combination hereof. Preferably, the aggregated casein-containing material is selected from the group consisting of milk, whole milk, skimmed milk, milk concentrates, reconstituted milk powder, non-pasteurised milk, micro-filtrated milk, pH-adjusted milk. [omskrevet]

It is an aspect of the present invention, that the aggregated casein-containing material has not been subjected to casein precipitation or removal of casein micelles and/or the casein aggregates prior to separation of soluble proteins.

In yet an embodiment of the present invention the soluble proteins are separated directly from the aggregated casein-containing material.

In the present context the term “casein aggregates” relates to micellar structured casein and aggregated caseins in various sizes. The micellar casein are provided when the casein molecules are formed as they begin folding up into a spherical micellar structure so that the casein proteins can remain suspended indefinitely in the milk water.

In another embodiment of the present invention, the aggregated casein-containing material comprises at least 5 g casein/l aggregated casein-containing material, such as at least 10 g casein/L aggregated casein-containing material, e.g. at least 15 g casein/L aggregated casein-containing material, such as at least 20 g casein/L aggregated casein-containing material, e.g. at least 22 g casein/L aggregated casein-containing material, such as at least 24 g casein/L aggregated casein-containing material, e.g. at least 30 g casein/L aggregated casein-containing material, such as at least 40 g casein/L aggregated casein-containing material, e.g. at least 50 g casein/L aggregated casein-containing material, such as at least 60 g casein/L aggregated casein-containing material, e.g. at least 70 g casein/L aggregated casein-containing material, such as at least 80 g casein/L aggregated casein-containing material, e.g. at least 90 g casein/L aggregated casein-containing material, such as at least 100 g casein/L aggregated casein-containing material.

Medium size scale production and/or industrial scale production may be performed in a batch process. Preferably, such batch process involves processing at least 50 litres aggregated casein-containing material per cycle, such as at least 100 litres aggregated casein-containing material per cycle, e.g. 250 litres aggregated casein-containing material per cycle, such as at least 500 litres aggregated casein-containing material per cycle, e.g. 750 litres aggregated casein-containing material per cycle, such as at least 1,000 litres aggregated casein-containing material per cycle, e.g. 2,500 litres aggregated casein-containing material per cycle, such as at least 5,000 litres aggregated casein-containing material per cycle, e.g. 7,500 litres aggregated casein-containing material per cycle, such as at least 10,000 litres aggregated casein-containing material per cycle, e.g. 25,000 litres aggregated casein-containing material per cycle, such as at least 50,000 litres aggregated casein-containing material per cycle, e.g. 75,000 litres aggregated casein-containing material per cycle, such as at least 100,000 litres aggregated casein-containing material per cycle, e.g. 250,000 litres aggregated casein-containing material per cycle.

Alternatively, large-scale production (industrial scale production) may be conducted at a continuous process. When the soluble proteins are retained by the chromatographic support an elution step at some point during the separation process may be necessary. By providing at least two chromatographic supports and placing them in parallel, such continuous separation may be provided where the flow of aggregated casein-containing material may be shifted from ore chromatographic support, when this chromatographic material is loaded and ready for elution, to the other chromatographic support. Alternatively, moving bed chromatography, simulated moving bed chromatography or the like may be used.

In an embodiment of the present invention, the continuous separation may have a capacity of at least 5,000 litres aggregated casein-containing material per hour, such as at least 10,000 litres aggregated casein-containing material per hour, e.g. at least 12,000 litres aggregated casein-containing per hour, such as at least 15,000 litres aggregated casein-containing material per hour, e.g. at least 18,000 litres aggregated casein-containing per hour, such as at least 20,000 litres aggregated casein-containing material per hour, e.g. at least 25,000 litres aggregated casein-containing per hour, such as at least 50,000 litres aggregated casein-containing material per hour, e.g. at least 100,000 litres aggregated casein-containing per hour.

In an embodiment of the present invention, conductivity and/or pH of the aggregated casein-containing material does not need to be adjusted before adding the aggregated casein-containing material to the chromatographic support. Hence, the pH and/or the conductivity of the aggregated casein-containing material may be as the aggregated casein-containing material as originally provided.

In an embodiment of the present invention, the aggregated casein-containing material may comprise minerals. In a preferred embodiment of the present invention, the aggregated casein-containing material has not been subjected to removal and/or addition of minerals. In a preferred embodiment of the present invention the aggregated casein-containing material comprise the minerals naturally present.

Preferably, the mineral is selected from the group consisting of calcium, phosphorus, iodine, magnesium, zinc, and potassium. Preferably, the mineral(s) present in the whey material is/are naturally present in the whey material.

In the present context the, term “naturally present” relates to the minerals present in the aggregated casein-containing material and are not a separately added compound, but found in the aggregated casein-containing material provided in step (i).

In an embodiment of the present invention, the aggregated casein-containing material has not been subjected to pasteurisation.

### Chromatographic Support

As described above, the present invention teaches the use of a chromatographic support allowing one or more soluble protein(s) from the aggregated casein-containing material to be retained, step (ii).

In the present context, the term “chromatography support” relates to any kind of container comprising an adsorbent, which can be supplied with at least one inlet for the application of the aggregated casein-containing material and at least one outlet for obtaining the at least one soluble protein fraction when subjected to an elution buffer.

The chromatographic support to be used may be a membrane chromatography support, preferably a mixed mode membrane chromatography support, or a column chromatography support. Preferably, the column chromatography support includes a Packed Bed Chromatography, stirred tank adsorption, moving bed chromatography, simulated moving bed chromatography, Fluidized Bed Chromatography and/or Expanded Bed Chromatography.

The fact that the Expanded Bed Chromatography (EBA) technology generally can work efficiently with non-clarified raw materials, such as an aggregated casein-containing material, makes it an attractive solution to implement for the isolation and separation of biomolecular substances. Compared to processes based on packed bed chromatography, Expanded Bed Chromatography may offer a robust process comprising fewer steps and thus results in increased yields and an improved process economy. Due to the expansion of the adsorbent bed during execution of an EBA process, EBA columns may further be scaled up to industrial scale without any significant considerations regarding increased back pressures or breakdown of the process due to clogging of the system which is often a problem when using packed bed columns. Hence, Expanded Bed Chromatography may be the preferred column chromatographic support according to the present invention.

Generally, the Expanded Bed Adsorption is well known to the person skilled in the art, and the method described in the present invention may be adapted to the processes described in WO 92/00799, WO 92/18237, WO 97/17132, WO 00/57982 or WO 98/33572.

In an embodiment of the present invention the aggregated casein-containing material may be loaded on to the chromatographic support at a flow-rate in the range of 1-50 cm/min; preferably in the range of 5-30 cm/min; more in the range of 10-25 cm/min; even more preferably, in the range of 15-20 cm/min.

### Adsorbent

In a preferred embodiment of the present invention, the chromatographic support may comprise an adsorbent.

Before the aggregated casein-containing material may be contacted with the adsorbent an initial, but optional, step in the method of the invention may involves equilibration of the adsorbent. Such equilibration may be done by using an equilibration liquid. PH of the equilibration liquid may vary dependent on the type of aggregated casein-containing material and/or the ligand used.

Equilibration of the adsorbent may preferably be done by using an acid or water, such as tap water, purified water, deionised water, demineralized water, or distilled water. In the event the equilibration liquid is an acid, the equilibration liquid may comprise low cost mineral acids such as hydrochloric acid, phosphoric acid, sulphuric acid. However, food grade organic acids such as acetic, citric and lactic acid may also be particularly preferred.

In the present context the term “adsorbent” relates to the entire bed present in the chromatographic support and is responsible for retaining the one or more soluble protein(s).

In an embodiment of the present invention, the adsorbent may comprise individual particles. In the present context, the term “adsorbent particle” is used interchangeably with the term “particle” and relates to the individual single particles which makes up the adsorbent.

In another embodiment of the present invention, the adsorbent may comprise a membrane coupled with a mixed mode ligand capable of binding the one or more soluble protein(s).

In the event the adsorbent, in the form of particles, is used in Expanded bed Adsorption several features, such as the flow rate, the size of the particles and the density of the particles may have influence on the expansion of the fluid bed and the separation of the proteins. It is important to control the degree of expansion in such a way to keep the adsorbent particles inside the column, but at the same time optimize the flow rate.

The degree of expansion may be determined as H/H0, where “H0” is the height of the bed In packed bed mode and “H” is the height of the bed in expanded mode. In an embodiment of the present invention, the degree of expansion H/H0 is in the range of 1.1-10 e.g. 1.0-6, such as 1.2-5, e.g. 1.3-5, such as 1.5-4, e.g. 4-6, such as 3-5, e.g. 3-4, such as 4-6.

In another embodiment of the present invention the degree of expansion H/H0 is at least 1.1, such as at least 1.5, e.g. at least 2, such as at least 2.5, e.g. at least 3, such as at least 3.5, e.g. at least 4, such as at least 4.5, e.g. at least 5, such as at least 5.5, e.g. at least 6, such as at least 10.

Furthermore, the density of the EBA adsorbent particle may be highly significant for the applicable flow rates in relation to the maximal degree of expansion of the adsorbent bed possible inside a typical EBA column (e.g. H/H0 max 3-5) and must be at least 1.3 g/ml, more preferably at least 1.5 g/ml, still more preferably at least 1.8 g/ml, even more preferably at least 2.0 g/ml, most preferably at least 2.3 g/ml, in order to enable a high productivity of the method.

The density of the EBA adsorbent particle is meant to be the density of the adsorbent particle in it's fully solvated (e.g. hydrated) state as opposed to the density of a dried adsorbent particle.

In an embodiment of the present invention the adsorbent particle has a mean particle size of at most 250 μm, such as at most 200 μm, e.g. at most 180 μm, particularly such as at most 160 μm, e.g. at most 150 μm, such as at most 140 μm, e.g. at most 130 μm, such as at most 120 μm, e.g. at most 110 μm, such as at most 100 μm, even more typically, the adsorbent particle has a mean particle size in the range of 90-250 μm, e.g. 100-200 μm, such as 120-180 μm, e.g. 140-160 μm.

It is to be understood that mean particle sizes below 100 μm such as below, 90 μm, e.g. below 80 μm, such as below 70 μm, e.g. below 60 μm, such as below 50 μm, e.g. below 40 μm, such as below 30 μm, e.g. below 20 μm, such as below 10 μm are also covered by the present invention. Using adsorbent particles having a mean particle size below 100 μm, however, may lead to lower productivity compared to using adsorbent particles having a mean particle size at or above 100 μm.

The high density of the adsorbent particle may be, to a great extent, achieved by inclusion of a certain proportion of a dense non-porous core materials, preferably having a density of at least 4.0 such as at least 10 g/ml, e.g. at least 16 g/ml, such as at least 25 g/ml. Typically, the non-porous core material has a density in the range of about 4.0-25 g/ml, such as about 4.0-20 g/m., e.g. about 4.0-16 g/ml, such as 12-19 g/ml, e.g. 14-18 g/ml, such as about 6.0-15.0 g/ml, e.g. about 6.0-16 g/ml.

The adsorbent particle used according to the present invention may be at least partly permeable to the proteins present in the aggregated casein-containing material in order to ensure a significant binding capacity in contrast to impermeable particles that can only bind the target molecule on its surface resulting in relatively low binding capacity, The adsorbent particle may be of an array of different structures, compositions and shapes.

The adsorbent particles may be constituted by a number of chemically derivatised porous materials having the necessary density and binding capacity to operate at the given flow rates per se. The particles may be either of the conglomerate type, as described in WO92/00799, having at least two non-porous cores surrounded by a porous polymeric base matrix, or of the pellicular type having a single non-porous core surrounded by a porous polymeric base matrix.

The adsorbent may comprise a porous polymeric base matrix having the one or more mixed-mode ligands covalently attached. Preferably, the porous polymeric base matrix may be a porous organic polymeric base matrix. In an embodiment of the present invention, the adsorbent may comprise a dense non-porous core material surrounded by the porous polymeric base matrix.

In the present context the term “conglomerate type” relates to a particle of a particulate material, which comprises high density non-porous core beads, having a core material of different types and sizes, held together by porous polymeric base matrix, e.g. a core particle consisting of two or more high density particles held together by surrounding agarose (porous polymeric base matrix).

In the present context, the term “pellicular type” relates to a composite of particles, wherein each particle consists of only one high density core material coated with a layer of porous polymeric base matrix, e.g. a high density stainless steel bead coated with agarose.

Accordingly, the term “at least one high density non-porous core” relates to either a pellicular core, comprising a single high density non-porous particle or it relates to a conglomerate core comprising more than one high density non-porous particle.

In the present context, the term “core” relates to the core particles present inside the adsorbent. The core particle or core particles may be incidentally distributed within the porous polymeric base matrix and is not limited to be located in the centre of the adsorbent.

In an embodiment of the present invention, the non-porous core constitutes typically of at most 50% of the total volume of the adsorbent, such as at most 40%, e.g. at most 30%, such as at the most 25%, e.g. at the most 20%, such as at the most 10%, e.g. at the most 5%.

In another embodiment of the present invention, the pore volume, accessible for proteins having a molecular weight higher than 1000 Dalton, constitute at least 50% of the adsorbent, such as at least 60%, e.g. at least 70%, such as at least 75%, e.g. at least 80%, such as at least 90% of the adsorbent.

The person skilled in the art knows various non-porous core materials and various porous polymeric base matrix. Examples of non-porous core materials and porous polymeric base matrixes may be found in WO 2010/037736. The skilled person also knows methods of preparing the adsorbent according to the present invention, such methods of preparing the adsorbent may be described in WO 2010/03776, EP 0 538 350 or WO 97/17132.

In an embodiment of the present invention, the polymeric base matrix does not comprise a styrene vinyitriethoxysilane copolymer.

In a further embodiment of the present invention, the adsorbent does not comprise silica particles or alumina particles. Preferably, the adsorbent does not comprise silica particles, alumina particles or ceramic particles coated with a styrene vinyltriethoxysilane copolymer.

During operation, the aggregated casein-containing material may be contacted with the adsorbent and the one or more soluble protein(s) may be adsorbed or fixated to the adsorbent whereas other components, such as aggregated casein, minerals, carbohydrate or combinations hereof, does not bind to the chromatographic support and run through the adsorbent. This adsorption may be performed under pressure. Particulate and/or unbound material and soluble impurities are optionally removed from the column during an optional washing.

When contacting the aggregated casein-containing material with the adsorbent, the ratio between the adsorbent and the aggregated casein-containing material may be optimized in order to provide a high capacity of the adsorbent and to obtain a high purity, high yield and/or high recovery of the at least one soluble protein fraction to be isolated.

In an embodiment of the present invention, the chromatographic support may have a loading ratio of soluble protein relative to the adsorbent of at least 10 mg soluble protein loaded per ml adsorbent, such as at least 12 mg, e.g. at least 15 mg, such as at least 20 mg, e.g. at least 25 mg, such as at least 30 mg, e.g. at least 35 mg, such as at least 50 mg, e.g. at least 75 mg, such as at least 100 mg, e.g. at least 150 mg, such as at least 175 mg, e.g. at least 200 mg.

In another embodiment of the present invention, the loading ratio of the aggregated casein-containing material relative to the adsorbent is at least 50 mg aggregated casein loaded per ml adsorbent, such as at least 75 mg, e.g. at least 100 mg, such as at least 200 mg, e.g. at least 300 mg, such as at least 400 mg, e.g. at least 500 mg, such as at least 600 mg, e.g. at least 700 mg, such as at least 800 mg, e.g. at least 900 mg, such as at least 1000 mg, e.g. at least 1500 mg.

In the present context, the adsorbent is in its fully solvated (e.g. hydrated) state when determining the loading ration of the soluble protein and/or the aggregated casein-containing material.

### Ligand

In order for the chromatographic support and/or the adsorbent to retain one or more soluble protein(s) from the aggregated casein-containing material, the adsorbent may comprise a ligand.

In a preferred embodiment of the present invention, the adsorbent may comprise one or more ligands having affinity for one or more soluble protein(s) present in the aggregated casein-containing material.

In the present context, the term “ligand” relates to a compound covalently attached to the adsorbent and which possesses the adsorbing function of one or more soluble protein(s).

The ligand may be a low molecular weight compound and in an embodiment of the present invention the ligand may have a molecular weight of at the most 1000 Dalton, such as at most 750 Dalton, e.g. at most 500 Dalton, such as at the most 250 Dalton, e.g. at the most 100 Dalton, e.g. at the most 50 Dalton.

In an embodiment of the present invention, the ligand is a chemically stabile ligand when present in an alkaline medium and/or the adsorbent is a chemically stabile adsorbent in an alkaline medium. The advantage of having a chemically stabile ligand and/or a chemically stabile adsorbent is that leakage of ligand moieties into one or more fractions may be reduced; possible toxicity issues may be avoided or reduced and/or the high performance of the chromatographic support may be maintained. In a preferred embodiment of the present invention, the chromatographic support maintain at least 75% of the ligand concentration upon incubation in 1M NaOH at 37° C. for 3 days in the dark, such as at least 80%, e.g. at least 85%, such at least 90%, e.g. at least 95%.

In yet an embodiment of the present invention, the ligand is coupled to the chromatographic support by a covalent binding, preferably, a strong covalent binding providing the chemically stabile ligand and/or the chemically stabile adsorbent.

In an embodiment of the present invention, the strong covalent binding may be provided by including one or more activating agents between the adsorbent and the ligand. Examples of such suitable activating reagents include epichlorohydrin, epibromohydrin allyl-glycidylether; bis-epoxides; halogen-substituted aliphatic compounds; aldehydes; quinones; chloro-triazines; oxazolones; and maleimides. Preferred activating reagents are epoxy-compounds, such as epichlorohydrin, allyl-glycidylether and butanedioldiglycidylether.

In the context of the present invention, the term “mixed mode ligand” relates to ligands having multiple behaviour, e.g. when operating mixed-mode ligands the chromatographic behaviour is based on a combination of e.g. electrostatic and hydrophobic properties of the protein and ligands.

In the present context, the term “multiple behaviour” relates to the capability of the ligand to interact with the soluble proteins through multiple types of molecular interactions such as hydrophobic interaction, ionic interaction, n-n interaction, van der Waals interaction, etc.

The mixed-mode ligand according to the present invention may comprise at least one hydrophobic moiety and at least one aromatic nitrogen moiety.

In a preferred embodiment of the present invention the chromatographic support comprises a porous organic polymeric base matrix having one or more mixed-mode ligands covalently attached, said one or more mixed-mode ligands comprise a hydrophobic moiety and a non-aromatic nitrogen moiety.

In an embodiment of the present invention, the ligand further comprises at least one chargeable moiety. Preferably, the mixed-mode ligand according to the present invention comprises at least one hydrophobic moiety, at least one chargeable moiety and at least one non-aromatic nitrogen moiety.

In an embodiment of the present invention the at least one chargeable moiety is an alkaline moiety having a pKa value of at most 11.0, such as at most 10.0, e.g. at most 9.0, such as at most 8.0, e.g. at most 7.0, such as at most 6.0, e.g. at most 5.0.

The at least one chargeable moiety may be a fully or partly positively charged moiety (at the pH equal to the pKa value, the moiety would be 50% charged) at the pH of the aggregated casein-containing material, such as milk, preferably at a pH value in the range of pH 6-8.

In an embodiment of the present invention, the chargeable moiety may be a carboxylic acid or an amine, selected from the group consisting of a primary amino-group, a secondary amino-group, a tertiary amino-group and a quaternary amino-group.

In yet an embodiment of the present invention the hydrophobic moiety may be an alkyl moiety or an optionally substituted, aromatic or heteroaromatic moiety or a combination of an alkyl-aromatic or heteroaromatic moiety.

In the event the hydrophobic moiety is an alkyl moiety the alkyl moiety may be an unbranched alkyl moiety. In a further embodiment of the present invention the alkyl moiety may comprise at least 3 carbon atoms, such as at least 4 carbon atoms, e.g. at least 5 carbon atoms.

In another embodiment of the present invention the ratio between nitrogen in the non-aromatic nitrogen moiety and carbon in the alkyl moiety is at least 1:3, such as at least 1:4, e.g. at least 1:5, as at least 1:6, e.g. at least 1:7, such as at least 1:8, e.g. at least 1:9, such as at least 1:10.

In the context of the present invention, the ratio between nitrogen and carbon may be defined by the number of atoms, e.g. a ratio between non-aromatic nitrogen and carbon of at least 1:3 means 1 non-aromatic nitrogen atom relative to at least 3 carbon atoms.

In an embodiment of the present invention, the chargeable moiety forms part of the non-aromatic nitrogen moiety.

In another embodiment of the present invention, the non-aromatic nitrogen moiety may be at least one nitrogen atom, at least one primary amino group, at least one secondary amino group, at least one tertiary amino-group, or at least one quaternary amino-group.

In an embodiment of the present invention, the ligand comprises a chargeable moiety or a chargeable moiety which forms part of the non-aromatic nitrogen group. The chargeable moiety may be charged at any pH values or the chargeable moiety may be chargeable at any pH value. In the present invention, the chargeable moiety being charged at any pH values may be a quaternary amino-group. In yet an embodiment of the present invention the chargeable moiety being chargeable at any pH values may be a primary amino-group, a secondary amino-group, or a tertiary amino-group.

In order to improve capacity, purity and recovery the ligand concentration may also be important. Hence, in a preferred embodiment of the present invention the ligand concentration may be in the range of 20-300 μmoles per ml sedimented adsorbent, e.g. 25-100 μmoles per ml sedimented adsorbent, such as 30-80 μmoles per ml sedimented adsorbent, e.g. 40-70 μμmoles per ml sedimented asorbent, e.g. 50-200 μmoles per ml sedimented adsorbent, such as 75-175 μmoles per ml sedimented adsorbent, e.g. 100-160 μmoles per ml sedimented asorbent, such as 120-145 μmoles per sedimented adsorbent.

In the present context, the terms “sedimented adsorbent” or “adsorbent particle” means an adsorbent in it's fully solvated (e.g. hydrated) state as opposed to the ligand concentration of a dried adsorbent.

### The Permeate Fraction

When contacting the aggregated casein-containing material with the chromatographic support one or more soluble protein(s) may be retained by the chromatographic support, whereas the aggregated casein remains unbound, runs through the chromatographic support and may be found in the permeate fraction.

The permeate fraction may further comprise at least one compound selected from the group consisting of lactoferrin, lactoperoxidase, aggregated casein, mineral, vitamin, carbohydrate and fat.

The minerals may be selected from the group consisting of calcium, phosphorus, iodine, magnesium, zinc and potassium. Even more preferably, the mineral may be calcium and a second mineral selected from the group consisting of phosphorus, iodine, magnesium, zinc and potassium.

The minerals present in the permeate fraction may preferably be mineral(s) from the aggregated casein-containing material. Preferably, 50% of the minerals in the permeate fraction comes from the aggregated casein-containing material, e.g. such as at least 75% of the minerals in the permeate fraction comes from the aggregated casein-containing material, such as at least 90% of the minerals in the permeate fraction comes from the aggregated casein-containing material, e.g. at least 92% of the minerals in the permeate fraction comes from the aggregated casein-containing material, such as at least 95% of the minerals in the permeate fraction comes from the aggregated casein-containing material, e.g. at least 98% of the minerals in the permeate fraction comes from the aggregated casein-containing material, such as 100% of the minerals in the permeate fraction comes from the aggregated casein-containing material.

In an embodiment of the present invention at least 20% (w/vv) of the minerals present in the aggregated casein-containing material relative to the total amount of minerals in the aggregated casein-containing material are present in the permeate fraction, such as at least 30%, e.g. at least 40%, such as at least 50% e.g. at least 70%, such as at least 80% e.g. at least 90%, such as at least 95%, e.g. at least 98%, such as at least 99% e.g. at least 99.5%, such as at least 99.9%.

The permeate fraction may be subjected to a further fractionation step. Preferably, lactoferrin and/or lactoperoxidase are separated from the permeate fraction, Methods for separating lactoferrin and/or lactoperoxidase, e.g. from the permeate fraction, are well known to the person skilled in the art.

In an embodiment of the present invention the yield of lactoferrin obtained from the further fractionation step is at least 50% of the amount of lactoferrin present in the aggregated casein-containing material and/or in the permeate fraction, such as at least 75%, e.g. at least 80%, such at least 85%, e.g. at least 90%, such at least 95%, e.g. at least 97%, such at least 99%.

In an embodiment of the present invention, the lactoferrin fraction separated according to the present invention may be used, alone or in a composition, in applications known to the skilled person. In particular, the lactoferrin fraction may be used, alone or in a composition, for infant formulas, dairy products, sports and fitness nutrition, pharmaceutical products, nutraceutical products, therapeutical products, weight management, ready-to-eat hot meals, processed meat, cosmetics, oral hygiene products or animal feed products.

In a further embodiment of the present invention the yield of lactoperoxidase obtained from the further fractionation step is at least 50% of the amount of lactoperoxidase present in the aggregated casein-containing material and/or in the permeate fraction, such as at least 75%, e.g. at least 80%, such at least 85%, e.g. at least 90%, such at least 95%, e.g. at least 97%, such at least 99%.

In an embodiment of the present invention, the lactoperoxidase fraction separated according to the present invention may be used, alone or in a composition, in applications known to the skilled person. In particular the lactoperoxidase fraction may be used, alone or in a composition, for diary products, soap products, such as shampoo, cosmetics, oral hygiene products, such as tooth paste, products against acne, plant protection products bactericide, or fungicide.

The further fractionation step removing lactoferrin and/or lactoperoxidase may be performed on the aggregated casein-containing material resulting in a lactoferring/lactoperoxidase depleted aggregated casein-containing material, In the present context, the term “lactoferring/lactoperoxidase depleted aggregated casein-containing material” relates to the presence of at most 0.05 mg/ml of the lactoferrin and/or lactoperoxidase, in a dry matter bases of the permeate fraction, such as at most 0.02 ml/mg, e.g. at most 0.01 ml/mg, such as at most 0.005 mg/ml.

In the event the aggregated casein-containing material provided in step (i) of the present invention is a lactoferring/lactoperoxidase depleted aggregated casein-containing material the permeate fraction comprises casein aggregates, mineral, vitamin, carbohydrate and fat.

In an embodiment of the present invention the permeate fraction, the second permeate fraction and/or the lactoferring/lactoperoxidase depleted aggregated casein-containing material may be used for manufacturing of a broad range of dairy products such as cheese, yoghurt, drinking milk products, fermented milk products, bakery, sweets and creamers. In particular, products that benefit from complete depletion or partial depletion of beta-lactoglobulin are preferred, such as infant formulas, hypo-allergenic products, and certain types of cheese known to the skilled person.

The further fractionation step results in at least one retained lactoferrin and/or lactoperoxidase fraction, two fractions comprising the individual constituents or a fraction comprising the combination of both, and a second permeate fraction comprising casein aggregates. The permeate fraction and/or the second permeate fraction may subsequently be subjected to curd formation and e.g. used for the production of cheese.

In an embodiment of the present invention the curd formation may be provided by the addition of rennet to the permeate fraction and/or the second permeate fraction resulting in the formation of a curd fraction and a glycomacropeptide fraction (GMP-fraction). After the addition of rennet to the permeate fraction and/or the second permeate fraction the casein aggregates starts to precipitate forming a solid curd fraction and a glycomacropeptide fraction excreted into the liquid phase. The curd fraction and the GMP-fraction may be separated by filtration, centrifugation and/or decanting.

In another embodiment of the present invention, the curd formation may be provided by the addition of an acid or by the addition of an acid in combination with heat to the permeate fraction and/or the second permeate fraction resulting in formation of a solid curd fraction.

The cheese production process may follow traditional cheese production processes known to the skilled person. During traditional cheese production, determination of proper firmness of the precipitated casein is important before cutting the precipitated casein in order to drain the whey. Traditionally, cheese makers carries out a series of tests to identify the optimum firmness for cutting. In an embodiment of the present invention, the step of cutting the precipitated casein in order to drain the whey may be omitted or significantly reduced.

The advantage of using precipitated casein obtained from the present invention in cheese manufacturing is that the permeate fraction and/or the second permeate fraction is depleted or substantially depleted in plasminogen. This depletion or substantial depletion results in more stabile casein structures and a more reliable and reproducible cheese production process.

In an embodiment of the present invention, the fraction comprising glycomacropeptide (GMP) may be further isolated, e.g. by membrane filtration, adsorption chromatography or a combination hereof.

In a further embodiment of the present invention the yield of glycomacropeptide (GMP) is at least 50% GMP relative to the total amount of protein, such as at least 75%, e.g. at least 80%, such at least 85%, e.g. at least 90%, such at least 95%, e.g. at least 97%, such at least 99%.

In the present context, the second permeate fraction is different from the permeate fraction. Preferably, the second permeate fraction differs from the permeate fraction in that at least one soluble protein, e.g. the soluble proteins lactoferrin and/or lactoperoxidase, has been removed and are substantially absent in the second permeate fraction.

In the present context the term “substantially absent” relates to a soluble protein originally present in the permeate fraction, such as at least one of the soluble proteins lactoferrin and/or lactoperoxidase, but which has been separated from the permeate fraction by the further fractionation step. In the present context the term “substantially absent” relates to at most 10% of the soluble protein, e.g. lactoferrin and/or lactoperoxidase, relative to the total amount of soluble protein, e.g. lactoferrin and/or lactoperoxidase, in the permeate fraction, e.g. at most 5%, such as at most 3%, e.g. at most 1%, such as at most 0.1%.

In an embodiment of the present invention, the second permeate fraction comprises at most 0.015 g lactoferrin/L second permeate fraction, such as at most 0.31 g lactoferrin/L second permeate fraction, e.g. at most 0.005 g lactoferrin/L second permeate fraction, such as at most 0.001 g lactoferrin/L second permeate fraction, e.g. at most 0.0005 g lactoferrin/L second permeate fraction.

In a further embodiment of the present invention, the second permeate fraction comprises at most 0.003 g lactoperoxidase/L second permeate fraction, such as at most 0.001 g lactoperoxidase/L second permeate fraction, e.g. at most 0.0005 g lactoperoxidase/L second permeate fraction, such as at most 0.00001 g lactoperoxidase/L second permeate fraction.

### Wash

After the aggregated casein-containing material has been contacted with the chromatographic support and the one or more soluble protein(s) have been allowed to bind to the adsorbent, the method according to the present invention may also involve an optional step of washing using a wash buffer. Hence, the method for providing the at least one separated soluble protein fraction may further comprise the step of:
(iv) optionally washing the chromatographic support

The step of washing the chromatographic support may be performed by using a wash buffer, whereby a wash fraction may be obtained.

Once the aggregated casein-containing material have been contacted with the chromatographic support, the chromatographic support may be washed using a wash buffer. The pH of the wash buffer may be dependent on the process, the soluble protein(s) to be isolated and the ligand used.

In a preferred embodiment of the present invention, the wash buffer may be an acid or water, such as tap water, purified water, deionized water, demineralized water, or distilled water. The acids applicable for adjusting the pH value of the wash buffer may be selected from low cost mineral acids such as hydrochloric acid, phosphoric acid and sulphuric acid but also from food grade organic acids such as acetic, citric and lactic acid.

In an embodiment of the present invention, the flow rate used for the washing step may be selected from the ranges outlined previously for loading the aggregated casein-containing material to the chromatographic support.

### Elution

In order to obtain the one or more soluble protein(s) retained by the chromatographic support, the chromatographic support may be subjected to an elution buffer.

In the context of the present invention, the term “elution buffer” relates to a composition capable of changing the conditions of the chromatographic support from adsorbing the soluble proteins of the present invention to the release and elution of the at least one soluble protein fraction according to the present invention.

In order to control elution it is possible to use a change in pH, a change in hydrophobicity, a change in conductivity (e.g. addition of salts) or a combination hereof.

Different elution strategies may be provided. Either all the retained proteins may be eluted simultaneously or alternatively the retained proteins may be eluted sequentially using a selective elution procedure. In the present invention, the retained soluble proteins may be eluted, simultaneously or sequentially by changing the pH, changing the conductivity. The change may be an instant change or a gradual change.

Preferably, the at least one soluble protein fraction may be eluted by changing the pH. In a preferred embodiment of the present invention, the pH of the elution buffer may facilitate optimal desorption of soluble protein adsorbed to the chromatographic support. In a preferred embodiment of the present invention, the soluble protein to be isolated is beta-casein and the elution buffer has a pH below 6.0. In another embodiment of the present invention the soluble proteins to be isolated are beta-lactoglobulin, alpha-albumin and/or immunoglobulin G using an elution buffer having a pH or 6.0 or above. In yet an embodiment of the present invention the soluble protein to be isolated is beta-casein using an elution buffer having a pH below 6.0 and the soluble proteins to be isolated are beta-lactoglobulin, alpha-albumin and/or immunoglobulin G using an elution buffer which has a pH of 6.0 or above.

In an embodiment of the present invention the elution buffer may comprise sodium hydroxide, potassium hydroxide, calcium hydroxide, ammonium hydroxide, ammonium formate, potassium phosphate, sodium phosphate, sodium citrate, sodium acetate, sodium carbonate or any combinations hereof. Preferably, the elution buffer comprises sodium hydroxide, potassium hydroxide, calcium hydroxide, ammonium hydroxide or any combination hereof is preferred.

### The Soluble Protein Fraction

Depending on the elution strategy provided, the at least one soluble protein fraction may be a single soluble protein fraction comprising all the retained soluble proteins, or the retained soluble proteins may be eluted in two or more distinct soluble protein fractions comprising individual soluble proteins or specific groups of soluble proteins.

The at least one soluble protein fraction in accordance with the present invention may comprise one or more soluble protein(s). In an embodiment of the present invention the one or more soluble protein(s) may be selected from the group consisting of immunoglobulin G; alpha-lactalbumin; beta-lactoglobulin; serum albumin; lactoperoxidase; lactoferrin; osteopontin; plasminogen, transferrin, proteose peptone, such as PP3, alkaline phosphatase, lipase and soluble casein, such as beta-casein.

In the event two or more of the retained soluble proteins are found in the same soluble protein fraction it may be preferred that the relative ratio between the soluble proteins retained by the chromatographic support is substantially the same as the relative ratio of the same soluble proteins in the aggregated casein-containing material.

In an embodiment of the present invention the at least one soluble protein fraction comprise a protein profile substantially similar to a protein profile of an aggregated casein-containing material. Said protein profile may include two or more soluble proteins selected from the group consisting of immunoglobulin; alpha-lactalbumin; beta-lactoglobulin; serum albumin; lactoperoxidase; lactoferrin; osteopontin; plasminogen, transferrin, proteose peptone, such as PP3, alkaline phosphatase, lipase and soluble casein, retained by the chromatographic support, such as 3 or more soluble proteins, e.g. 4 or more soluble proteins, such as 5 or more soluble proteins, e.g. 6 or more soluble proteins.

In the present context the terms “substantially the same” and “substantially similar” relate to a difference of at the most 10% between the amount of soluble proteins present in the at least on soluble protein fraction relative to the amount of the same soluble proteins in the aggregated casein-containing material, such as at most 5%, e.g. at most 3%, such as at most 1%.

In an embodiment of the present invention the yield of the one or more soluble protein(s) in the soluble fraction may be at least 50% of the amount of the soluble proteins present in the aggregated casein-containing material, such as at least 75%, e.g. at least 80%, such at least 85%, e.g. at least 90%, such at least 95%, e.g. at least 97%, such at least 99%.

In a preferred embodiment of the present invention at least the soluble proteins imrnunoglobulin G or beta-casein and at least one of alpha-lactalbumin and/or beta-lactoglobulin present in the aggregated casein-containing material may be retained by the chromatographic support.

The separation according to the present invention provides, a permeate fraction and retention, by the chromatographic support, of at least the soluble proteins immunoglobulin G or beta-casein and at least one of alpha-lactalbumin and/or beta-lactoglobulin.

In an embodiment of the present invention, the separation relates to a process of providing at least one soluble protein fraction comprising a mixture of soluble proteins, such as at least immunoglobulin G or beta-casein and at least one of alpha-lactalbumin and/or beta-lactoglobulin.

The at least one soluble protein fraction in accordance with the present invention may comprise at least one of the soluble proteins immunoglobulin G, or beta-casein and at least one of alpha-lactalbumin and/or beta-lactoglobulin.

In another embodiment of the present invention, the separation relates to the provision of two or more distinct soluble protein fractions comprising individual soluble protein fractions.

In the event, two soluble protein fractions are provided they may comprise:
one soluble protein fraction comprising a beta-lactoglobulin fraction and the other soluble protein fraction may comprise an immunoglobulin G fraction or a combined immunoglobulin G/alpha-lactalbumin fraction;
one beta-lactoglobulin fraction and the other soluble protein fraction may comprise an beta-casein fraction;
one combined beta-lactoglobulin/beta-casein fraction and the other soluble protein fraction may comprise an immunoglobulin G fraction; an alpha-lactalbumin fraction or a combined immunoglobulin G/alpha-lactalbumin fraction;
one soluble protein fraction comprising an immunoglobulin G fraction and the other soluble protein fraction comprise an alpha-lactalbumin fraction or a beta-lactoglobulin fraction or a combined alpha-lactalbumin/beta-lactoglobulin fraction; or
one soluble protein fraction comprising an alpha-lactalbumin fraction and the other soluble protein fraction comprise an immunoglobulin G fraction or a combined immunoglobulin G/beta-lactoglobulin fraction.

In an embodiment of the present invention the combined fractions, such as the combined immunoglobulin G/alpha-lactalbumin fraction; the combined beta-lactoglobulin/beta-casein fraction; the combined alpha-lactalbumin/beta-lactoglobulin fraction and/or the combined immunoglobulin G/beta-lactoglobulin fraction may be further fractionated into two separate protein fractions.

In the event 3 soluble protein fractions are provided one fraction may comprise the immunoglobulin G fraction; one fraction comprising the beta-lactoglobulin fraction; and one fraction comprising the alpha-lactalbumin fraction.

In the event 4 soluble protein fractions are provided one fraction may comprise the immunoglobulin G fraction; one fraction comprising the beta-lactoglobulin fraction; one fraction may comprise the beta-casein fraction and one fraction comprising the alpha-lactalbumin fraction.

In yet an embodiment of the present invention two soluble protein fractions may be obtained, a first soluble protein fraction comprising soluble casein, such as beta-casein, and a second soluble protein fraction comprising one or more soluble protein(s) selected from the group consisting of immunoglobulin G, alpha-lactalbumin, beta-lactoglobulin; serum albumin; lactoperoxidase; lactoferrin; osteopontin; plasminogen, transferrin, proteose peptone, such as PP3, alkaline phosphatase and lipase.

In another embodiment of the present invention, the soluble casein fraction, such as the beta-casein fraction, separated according to the present invention may be used, alone or in a composition, in applications known to the skilled person. The soluble casein fraction, and in particular the beta-casein fraction may in particular be used, alone or in a composition, for infant formulas; emulgator compositions; foaming agents; oral hygiene, such as tooth paste; enzyme activation, in particular nuclease activation; dairy products; a source of lysine and/or tryptophan, e.g. for animal feed, such as animal feed for pigs or poultry; cosmetics; functional food, dietary products; hair repair; rheology and/or viscosity lowering; or pharmaceutical or nutraceutical compositions for increasing mineral uptake in the intestine, hypotension, opioid activity, ACE-inhibitory activity, against RSV, against influenza.

In accordance with the above embodiment, it is preferred that the yield of soluble casein, such as beta-casein, obtained in the first soluble protein fraction is at least 10%, 20%, 30% 40%, 50% of the amount of soluble casein, such as beta-casein, present in the casein-containing material, such as at least 75%, e.g. at least 80%, such at least 85%, e.g. at least 90%, such at least 95%, e.g. at least 97%, such at least 99%.

In yet an embodiment of the present invention three soluble protein fractions are obtained, a first soluble protein fraction comprising, soluble casein, such as beta-casein, a second soluble protein fraction comprising beta-lactoglobulin, and a third soluble protein fraction comprising one or more soluble protein(s) selected from the group consisting of immunoglobulin G, alpha-lactalbumin, serum albumin; lactoperoxidase; lactoferrin; osteopontin; plasminogen, transferrin, proteose peptone, such as PP3, alkaline phosphatase, and lipase.

In accordance with the above embodiment, it is preferred that the yield of beta-lactoglobulin obtained in the second soluble protein fraction is at least 10%, 20%, 30% 40%, 50% of the amount of beta-lactoglobulin present in the casein-containing material, such as at least 75%, e.g. at least 80%, such at least 85%, e.g. at least 90%, such at least 95%, e.g. at least 97%, such at least 99%.

In another embodiment of the present invention, the beta-lactoglobulin fraction, separated according to the present invention may be used, alone or in a composition, in applications known to the skilled person. The beta-lactoglobulin fraction may in particular be used, alone or in a composition, for dairy products; beverages; ready-to-eat hot meals; processed meat; bakery; stabilizing products; or vitamin or mineral carrier.

In a further embodiment of the present invention four soluble protein fractions are obtained, a first soluble protein fraction comprising soluble casein, such as beta-casein, a second soluble protein fraction comprising beta-lactoglobulin, a third soluble protein fraction comprising immunoglobulin G, and a fourth soluble protein fraction comprising one or more soluble protein(s) selected from the group consisting of alpha-lactalbumin, serum albumin; lactoperoxidase; lactoferrin, osteopontin; plasminogen, transferrin, proteose peptone, such as PP3, alkaline phosphatase and lipase.

In accordance with the above embodiment the yield of immunoglobulin G obtained in the third soluble protein fraction is at least 10%, 20%, 30% 40%, 50% of the amount of 15 immunoglobulin G present in the casein-containing material, such as at least 75%, e.g. at least 80%, such at least 85%, e.g. at least 90%, such at least 95%, e.g. at least 97%, such at least 99%.

In another embodiment of the present invention, the immunoglobulin G fraction, separated according to the present invention may be used, alone or in a composition, in applications known to the skilled person. The immunoglobulin G fraction may in particular be used, alone or in a composition, for infant formulas; dairy products; sports and fitness nutrition; pharmaceutical or nutraceutical products; weight management; cosmetics; nutritional immunotherapy; or passive immunization.

In an even further embodiment of the present invention five soluble protein fractions are obtained, a first soluble protein fraction comprising soluble casein, such as beta-casein, a second soluble protein fraction comprising beta-lactoglobulin, a third soluble protein fraction comprising immunoglobulin G, a fourth soluble protein fraction comprising alpha-lactalbumin, and a fifth soluble protein fraction comprising one or more soluble protein(s) selected from the group consisting of serum albumin; lactoperoxidase; lactoferrin; osteopontin; plasminogen, transferrin, proteose peptone, such as PP3, alkaline phosphatase and lipase.

In accordance with the above embodiment, the yield of alpha-lactalbumin obtained in the fourth soluble protein fraction is at least 10%, 20%, 30% 40%, 50% of the amount of alpha-lactalbumin present in the casein-containing material, such as at least 75%, e.g. at least 80%, such at least 85%, e.g. at least 90%, such at least 95%, e.g. at least 97%, such at least 99%.

In another embodiment of the present invention, the alpha-lactalbumin fraction, separated according to the present invention may be used, alone or in a composition, in applications known to the skilled person. The alpha-lactalbumin fraction may in particular be used, alone or in a composition, for infant formulas; humanized baby food; nutritional products; sports and fitness nutritional products; dairy products; beverages; pharmaceutical and/or nutraceutical composition, e.g. for reduction of stress, opioid activity, regulation of cells growth, antiulcer activity, irnmunornodulation, anti-hypertensive, anti-diarrhoea, sleep disorders, mood disorders and stress/depression problems.

These high recoveries of soluble protein may preferably be obtained from a single contact between the aggregated casein-containing material and the chromatographic support. In the present context, the term “single contact” relates to contacting the aggregated casein-containing material only one time with the chromatographic support and without re-cycling the permeate fraction or the at least one soluble protein fraction to the chromatographic support to improve separation.

Other soluble proteins and different orders of elution of the soluble proteins may be obtained in the at least one soluble protein fractions depending of the composition of the elution buffer and the elution procedure.

In order to improve stability of the proteins in the aggregated casein-containing material the one or more soluble proteins retained by the chromatographic support may be plasminogen.

The permeate fraction obtained from removing plasminogen may preferably be used for cheese production, long-term milk, UHT-milk or UHT-cream.

In an embodiment of the present invention, the at least one soluble protein fraction obtained may be subjected to a second concentration step. Such second concentration step may include ultrafiltration, nanofiltration, microfiltration, adsorption chromatography, centrifugation or any combination hereof. The second concentration step may result in a second concentrated retentate fraction comprising one or more of the soluble proteins present in the at least one soluble protein fraction and a third concentrated permeate fraction mainly comprising water. In a further embodiment of the present invention the water obtained in the third concentrated permeate fraction may preferably be reused in the method according the present invention.

In an embodiment of the present invention, the at least one soluble protein fraction may be a liquid, a concentrate or a powder.

### Further Embodiments

In most of the prior art, denaturation of proteins are not considered an issue, and process conditions are implemented that may jeopardise the functionality of the native proteins.

The present invention may benefit from very gentle handling of the soluble proteins and it may preferably be desired that the native functionality/functionalities of the separated soluble proteins is maintained or substantially maintained.

Different conditions may cause denaturation of soluble proteins, and some proteins in the aggregated casein-containing material may be more sensitive than others. Examples of conditions that may cause denaturation may be exposure to pH values below 3 and above 11; high salt concentrations; heat; and chemicals.

Thus in order to avoid denaturation of the soluble proteins the aggregated casein-containing material has preferably not been subjected to pasteurisation.

Even though high temperatures should be avoided in order not to risk denature of the soluble proteins, the method according to the present invention may advantageously be conducted at temperatures above ambient temperature. In a further embodiment of the present invention at least one of the steps (ii) to (v) may be performed at a temperature above 25° C., such as above 27° C., e.g. above 30° C., such as above 35° C., e.g. above 40° C., such as above 45° C., e.g. about 50° C., such as in the range of 25-80° C., e.g. in the range of 30-70° C., such as in the range of 35-65° C., e.g. in the range of 40-60° C., such as in the range of 45-55° C.

The soluble beta-casein may be bound to the micellar casein. However, at low temperatures the beta-casein molecules may dissociate or partly dissociate from the micellar casein. Hence, in an embodiment of the present invention the temperature of the aggregated casein-containing material may be in the range of 1-10° C., such as in the range of 2-7° C., e.g. in the range of 3-5° C. in step (i) and/or (ii). Alternatively, the temperature of the aggregated casein-containing material may be kept low, i.e. at temperatures in the range of 1-10° C. as mentioned above, prior to being contacted with the chromatographic material, but elevated to higher temperatures, such as in the range of 25-80° C. as mentioned above, immediately before being contacted with the chromatographic support.

The purity, yield and recovery of the soluble proteins obtained from the aggregated casein-containing material as described in the present invention may be provided from a single cycle of the whey material through the chromatographic support.

In the present context, the term “single cycle” relates to only one time contact between the chromatographic support and the aggregated casein-containing material. The permeate fraction, the second permeate fraction or the at least one soluble protein fraction are not re-cycled to the chromatographic support in order to provide further separation of the constituents present in the fractions. in an embodiment of the present invention the at least one soluble protein fraction may be provided by having two chromatographic supports serially connected. Thus, a series of at least 2 chromatographic supports may be provided and where the first chromatographic support is loaded with the aggregated casein-containing material and the second chromatographic support is loaded with a run through fraction obtained from the first chromatographic support, the permeate fraction.

In an embodiment of the present invention the first chromatographic support may be over-loaded with soluble protein to be retained by the chromatographic support relative to what the mixed-mode ligand is capable of binding.

In an embodiment of the present invention such overload may preferably lead to retaining the soluble proteins immunoglobulin G and/or optionally alpha-lactalbumin on the second chromatographic support and retaining the soluble protein beta-lactoglobulin, and optionally beta-casein, on the first chromatographic support.

in a preferred embodiment of the present invention the at least one soluble protein fraction according to the present invention and/or the lactoferrin/lactoperoxidase fraction, the glycomacropeptide fraction and/or the curd fraction according to the present invention may be used as an ingredient in a food product, in a feed product, in a dietary product, in a pharmaceutical product, a nutraceutical product, in a therapeutic product, in a beverage product, in a skin care product; a cosmetic product; a product for nutritional immunotherapy; or a product for passive immunization.

It should be noted that embodiments and features described in the context of one of the aspects of the present invention or in one of the embodiment of the present invention also apply to the other aspects or other embodiments of the invention.

All patent and non-patent references cited in the present application, are hereby incorporated by reference in their entirety.

### EXAMPLES

### Example 1

Separation of beta-lactoglobulin directly from skimmed milk. The skimmed milk is non-pasteurised skimmed milk which has not been subjected to removal of casein.

Substantially all the soluble proteins in the skimmed milk are retained by the chromatographic support and aggregated casein, mineral, vitamin, carbohydrate and fat runs through the chromatographic support. The beta-lactoglobulin fraction is subsequently eluted from the chromatographic support and analysed.

### Raw Material

The raw material used is skimmed milk obtained from raw milk (bovine), non-pasteurized, and collected from a local farmer. The cream was removed from the raw milk by centrifugation. The pH of the skimmed milk was pH 6.58 and without any pH adjustment (the natural pH of the milk).

### Adsorbent

The adsorbent used was a mixed mode ligand, based on benzylamine.

The adsorbent is based on 5% agarose with 10% tungsten carbide particles incorporated, density of approximately 2.9 and particle size in the range of 40-250 μm. The mixed mode adsorbents are cross-linked with epichlorhydrine and coupled with benzylamine. Ligand concentration: approximately, 42 mmol mixed mode groups/L adsorbent.

### Process Parameters

13.2 litre of adsorbent (mixed-mode adsorbent) is packed in a chromatographic column having a diameter of 15 cm. The bed height in packed mode is 75 cm.

The adsorbent was equilibrated with 75 litres water.

132 litres raw material, skimmed milk, was loaded on the chromatographic column resulting in a load ratio of 1:10 (litre adsorbent litre raw material). Flow rate, gravity: 20 cm/min resulting in a two times bed expansion (to 150 cm expanded bed height).

The adsorbent is washed with 75 litres water.

The beta-lactoglobulin was eluted with 25 litres 20 mM NaOH. The eluate is neutralised with 1 M hydrochloric add. The other soluble proteins from the skimmed milk and bound to the chromatographic material may be sequentially eluted by changing the elution buffer.

The experiment is performed at 50° C.

### Results

The results showed that 20.08 mg beta-lactoglobulin/ml eluate was obtained and a total of 562 g beta-lactoglobulin was obtained. The chromatographic material (and the mixed-mode ligand) showed a capacity of 42.6 mg beta-lactoglobulin per ml adsorbent.

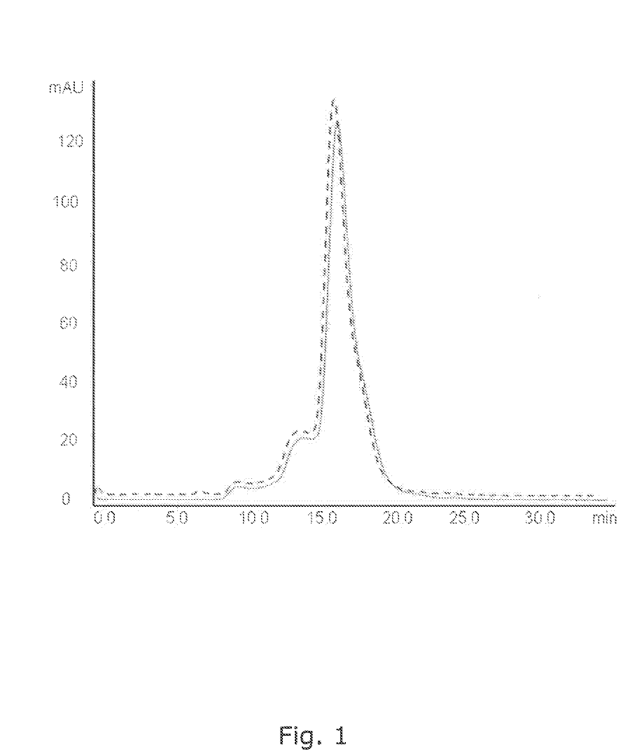

Gel filtration analysis demonstrated a highly pure beta-lactoglobulin fraction see FIG. 1, which shows the purity of the beta-lactoglobulin fraction obtained from the present example. The dashed line illustrates the purity of the beta-lactoglobulin fraction obtained directly from the chromatographic support, and the solid line illustrates the purity of beta-lactoglobulin fraction obtained from the chromatographic support followed by a subsequent step of micro filtration.

The yield of the different proteins in the various fractions (e.g. in the eluate (the beta-lactoglobulin fraction)) was estimated with SDS-PAGE technique. SDS-PAGE gel electrophoresis was performed according to the following general procedure:

25 μL of sample was mixed with 25 μL tris-glycine sample. buffer (LC2676, Novex by Life Technologies, USA). The resulting solution was boded in water for 5 min under non-reducing conditions. 20 μL of the boiled sample was loaded on to a precast SDS-PAGE gel cassette (4-20% tris-glycine gradient gel (1 mm), (EC6025, Novex by Life Technologies, USA). The gel was running for 1 hour at 200 V, 400 mA. The gel was stained with Coomassie blue dye reagent overnight (SimplyBlue™ SafeStain, LC6060).

The analysis based on the SDS-PAGE showed that the beta-lactoglobulin fraction comprise less than 5% (on a weight/weight basis) alpha-lactalbumin and only traces of immunoglobulins.

Thus, a beta-lactoglobulin fraction is provided which has a high purity, a high yield and a high recovery.

### REFERENCES

WO 92/00799

WO 92/18237

WO 97/17132

WO 00/57982

WO 98/33572

We claim:

1. A method for separating at least one soluble protein fraction from an aggregated casein-containing material, the method comprises the steps of:
(i) Providing the aggregated casein-containing material;
(ii) Contacting the aggregated casein-containing material with a chromatographic support allowing one or more soluble protein(s) present in the aggregated casein-containing material to be retained by the chromatographic support;
(iii) Obtaining a permeate fraction from the chromatographic support comprising aggregated casein;
(iv) Optionally washing the chromatographic support;
(v) Subjecting the chromatographic support to at least one elution buffer obtaining at least one soluble protein fraction from the chromatographic support; and
wherein the chromatographic support comprises one or more mixed-mode ligands capable of binding the soluble proteins from the aggregated casein-containing material.

2. The method according to claim 1, wherein the one or more soluble protein(s) is at least the soluble proteins immunoglobulin G or beta-casein and at least one of alpha-lactalbumin and/or beta-lactoglobulin present in the aggregated casein-containing material to be retained by the chromatographic support.

3.-14. (canceled)

15. The method according to claim 1, wherein two soluble protein fractions are obtained, a first soluble protein fraction comprising beta-casein, and a second soluble protein fraction comprising one or more soluble protein(s) selected from the group consisting of immunoglobulin G, alpha-lactalbumin, beta-lactoglobulin; serum albumin; lactoperoxidase; lactoferrin; osteopontin; plasminogen, transferrin, proteose peptone, such as PP3, alkaline phosphatase and lipase.

16. The method according to claim 1, wherein three soluble protein fractions are obtained, a first soluble protein fraction comprising beta-casein, and a second soluble protein fraction comprising beta-lactoglobulin, and a third soluble protein fraction comprising one or more soluble protein(s) selected from the group consisting of immunoglobulin G, alpha-lactalbumin, serum albumin; lactoperoxidase; lactoferrin, osteopontin; plasminogen, transferrin, proteose peptone, such as PP3, alkaline phosphatase and lipase.

17. The method according to claim 1, wherein four soluble protein fractions are obtained, a first soluble protein fraction comprising beta-casein, and a second soluble protein fraction comprising beta-lactoglobulin, a third soluble protein fraction comprising immunoglobulin G, and a fourth soluble protein fraction comprising one or more soluble protein(s) selected from the group consisting of alpha-lactalbumin, serum albumin; lactoperoxidase; lactoferrin; osteopontin; plasminogen, transferrin, proteose peptone, such as PP3, alkaline phosphatase and lipase.

18. The method according to claim 1, wherein five soluble protein fractions are obtained, a first soluble protein fraction comprising beta-casein, and a second soluble protein fraction comprising beta-lactoglobulin, a third soluble protein fraction comprising immunoglobulin G, and a fourth soluble protein fraction comprising alpha-lactalbumin, and a fifth soluble protein fraction comprising one or more proteins selected from the group consisting of serum albumin; lactoperoxidase; lactoferrin; osteopontin;
plasminogen, transferrin, proteose peptone, such as PP3, alkaline phosphatase and lipase,

19.-26. (canceled)

27. The method according to claim 1, wherein the native functionality/functionalities of the one or more soluble protein(s) present in the protein fraction have been maintained.

28. (canceled)

29. The method according to claim 1, wherein the aggregated casein-containing material has not been subjected to pasteurization.

30. The method according claim 1, wherein the aggregated casein-containing material has not been subjected to precipitation or removal of casein micelles prior to separation of soluble proteins.

31. The method according to claim 1, wherein the soluble proteins are separated directly from the aggregated casein-containing material.

32. The method according to claim 1, wherein the method is a selective elution process.

33.-34. (canceled)

35. The method according to claim 1, wherein the aggregated casein-containing material is loaded on to the chromatographic support at a flow-rate in the range of 1-50 cm/min; preferably in the range of 5-30 cm/min; more in the range of 10-25 cm/min; even more preferably, in the range of 15-20 cm/min.

36. (canceled)

37. The method according to claim 1, wherein a series of at least 2 chromatographic supports are provided and where the first chromatographic support is loaded with the aggregated casein-containing material and the second chromatographic support is loaded with a run through fraction, the permeate fraction, obtained from the first chromatographic support.

38. The method according to claim 37, wherein the first chromatographic support is over-loaded with aggregated casein-containing material and/or soluble protein to be retained by the chromatographic support relative to what the mixed-mode ligand is capable of binding.

39.-42. (canceled)

43. The method according to claim 1, wherein the permeate fraction obtained in step (iii) also comprise minerals, carbohydrates, fat, lactoferrin and/or lactoperoxidase.

44. The method according to claim 43, wherein the permeate fraction obtained in step (iii) may be subjected to a further fractionation step, and a second fractionation step results in at least one lactoferrin/lactoperoxidase fraction and a second permeate fraction, and wherein the second permeate fraction comprises aggregated casein.

45-47. (canceled)

48. The method according to claim 44, wherein a rennet is added to the second permeate fraction resulting in the formation of a curd fraction and a glycomacropeptide fraction (GMP-fraction).

49. (canceled)

50. The method according to claim 48, wherein the curd fraction is used in a cheese production.

51. A soluble protein fraction obtained by a method according to claim 1.

52.-62. (canceled)

63. A method of separating at least one soluble protein fraction from an aggregated casein-containing material, said method comprises the step of contacting the aggregated casein-containing material with a chromatographic support comprising a porous organic polymeric base matrix having one or more ligands covalently attached for separating the at least one soluble protein fraction from the aggregated casein-containing material.
